(19) United States
(12) Patent Application Publication
(10) Pub. No.: US 2010/0292325 A1
(43) Pub. Date: Nov. 18, 2010

(54) P-MENTHAWE-3-CARBOXYLIC ACID ESTERS TO TREAT AIRWAYS DISEASES

(76) Inventor: Edward Tak Wei, Berkeley, CA (US)

Correspondence Address: GlaxoSmithKline;GLOBAL PATENTS -US, UW2220, P. O. BOX 1539, KING OF PRUSSIA, PA, 19406-0939, US

(21) Appl. No.: 12/809,703

(22) PCT Filed: Dec. 17, 2008
(86) PCT No.: PCT/GB2008/004176

§ 371 Date: Jun. 21, 2010

Related U.S. Application Data
(60) Provisional application No. 61/008,980, filed on Dec. 21, 2007.

(51) Int. Cl. A61K 31/215; C07C 69/757; A61P 11/06; A61P 11/14; A61P 11/00; A61P 29/00; A61M 11/00

(52) U.S. Cl. 514/529; 560/1; 128/200.14

(57) ABSTRACT

The present invention relates to methods and devices for treating sensory discomfort in the upper airways of a human; treating sensory discomfort in the oropharynx of a human; alleviating pain from pharyngitis in a human; alleviating cough in a human; and ameliorating the symptoms and signs of asthma, dyspnea, sleep apnea, snoring or chronic obstructive pulmonary disease in a human. According to the invention, an active compound is delivered, preferably selectively delivered, to the oropharynx, preferably the oropharyngeal surfaces, more preferably the lower retropalatal oropharynx (LRO) of the patient. According to the invention, the active compound is a compound of the following formula, wherein R is C₂ to C₄ hydroxyalkyl or polyhydroxyalkyl.

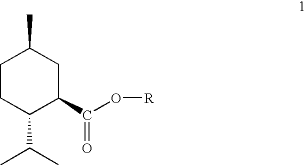

### RELATED APPLICATION

This application is related to U.S. Provisional Application No. 61/008,980 filed 21 Dec. 2007, the contents of which are incorporated herein by reference in their entirety.

### TECHNICAL FIELD

The present invention relates to methods and devices for treating sensory discomfort in the upper airways of a human; treating sensory discomfort in the oropharynx of a human; alleviating pain from pharyngitis in a human; alleviating cough in a human; and ameliorating the symptoms and signs of asthma, dyspnea, sleep apnea, snoring, or chronic obstructive pulmonary disease in a human. According to the invention, an active compound is delivered, preferably selectively delivered, to the oropharynx, preferably the oropharyngeal surfaces, more preferably the lower retropalatal oropharynx (LRO) of the patient. According to the invention, the active compound is a compound of the following formula, wherein —R is C₂ to C₄ hydroxyalkyl or polyhydroxyalkyl:

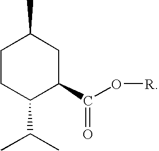

### BACKGROUND

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a carrier” includes mixtures of two or more carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

### Airway Irritation and Cough

Airway irritation initiates the sensations that lead to cough. The causes of airway irritation and obstruction are multi-factorial and include conditions such as viral or bacterial upper airway infections, post-nasal drip, allergies, inflammation of the airways from air pollutants, pharyngitis, laryngitis, and for chronic coughing such conditions as asthma, chronic obstructive lung disease, gastroesophageal reflux disease, lung cancer, pneumonia, sleep apnea, snoring, pulmonary edema, congestive heart failure, and dyspnea.

Current medications for coughing have limited efficacy, as witnessed by individuals who stay awake at night, unable to sleep because of cough, and individuals who cough for prolonged periods, for example, for 3 weeks after a viral infection of the upper airways.

There is need for a new medication, simply applied, that will control cough for at least three to four hours to allow the patient to stop coughing and go to sleep.

Cough is a reflex designed to remove sensory irritants and obstructions from the airways. The origin of the stimuli for cough is generally felt to come from the larynx (voice box) although the actual sites of inflammation that generates the cough signal may originate from the esophagus and from the bronchi. Coughing is a familiar experience and is executed by a coordinated contraction of the respiratory muscles against a closed glottis.

### Existing Drugs

A number of drugs are available on the market for the treatment of cough. Their mode of action and/or method of delivery is different from that of the present invention.
Dextromethorphan and codeine are “centrally-acting anti-tussives”, i.e., they are thought to act on elements in the brain or spinal cord to suppress cough. Dextromethorphan, in most individuals, is rapidly metabolized after “first-pass” absorption via the gastrointestinal tract. Hence, it must be taken 3 or 4 times a day to maintain an adequate plasma level. Dextromethorphan is used in more than 150 over-the-counter preparations for cough. Codeine is a Schedule 3 drug that can only be obtained by prescription. It belongs to the class of drugs known as narcotic analgesics. Both codeine and dexthromethorphan are subject to abuse. The efficacy of dextromethorphan and codeine in suppressing cough has been questioned. In double-blind, placebo-controlled studies, both drugs were not better than placebo. On the other hand, placebos, especially active placebos such as sugary syrups, are sometimes effective in the suppression of cough.
First generation antihistamines, such as Benadryl® (diphenhydramine) and Chlortrimeton® (chlorpheniramine) have a drying effect on nasal secretions which are sometimes beneficial for cough associated with the common cold and allergies. These compounds also have a sedative, depressive action on the brain. Antihistamines are not effective for dry hacking coughs seen, for example, in flus or asthma. Side-effects such as sedation and dry mouth limit the use of antihistamines for the treatment of cough.
Guaifenesin is an “expectorant” which means it promotes the secretion and “thinning” of mucus on the surface of the airways. The efficacy of guaifenesin in various forms of cough has not been established in placebo-controlled, double-blind studies. Guaifenesin is an ingredient in many generic products. Hypersecretion of mucus is contraindicated in certain patients with asthma or chronic obstructive pulmonary disease (COPD) and guaifenesin should not be used in such patients.
Menthol cough drops or lozenges typically weigh about 3.4 g (Walgreen's cough drops) or 2.7 g (N′Ice lozenges) and contain from 5, 7, or up to a maximum of 10 mg of (−)-menthol in a sugar-dye matrix. Doses of menthol higher than 7 mg are less common because the harsh taste of (−)-menthol makes the lozenge unpalatable. The menthol lozenges are held in mouth for about 10 to 15 minutes and soothe the lining of the mouth and the throat. A drawback to long term use of lozenges is the addition of sugar and calories to the diet.

### Delivery of Drugs and Suppression of Cough

Drugs such as dextromethorphan, codeine, and antihistamines must be delivered via the bloodstream to neuronal receptors. Hence, these drugs are usually administered as pills or dissolved in syrups and have to be absorbed into the systemic circulation.

A safer mode of drug administration is to use topical application of a drug onto the throat. Locally delivery of anti-tussive agents into the oral cavity has been proposed using soft and chewable compositions which contain one or more active ingredients (see, e.g., Cherukuri et al., 2000, WO 00/37044 A1).

Another approach is to administer medicaments into the upper respiratory tract using binding agents or edible film incorporating active anti-cough medications (see, e.g., Pan et al., 1999, U.S. Pat. No. 5,912,007).

### SUMMARY OF THE INVENTION

### Methods of Treatment

One aspect of the present invention relates to methods of treatment including, for example:
A method of treating sensory discomfort in the upper airways of a human.
A method of treating sensory discomfort in the oropharnyx of a human.
A method of alleviating pain from pharyngitis in a human.
A method of alleviating cough in a human.
A method of ameliorating the symptoms and signs of asthma, dyspnea, sleep apnea, snoring, or chronic obstructive pulmonary disease in a human.

The treatment comprises administering to the human in need of treatment a therapeutically-effective amount of an active compound, by delivering the active compound, for example:
to the oropharynx of the human.
more preferably, onto the oropharyngeal surfaces of the human.
still more preferably, to the lower retropalatal oropharynx (LRO) of the human.

Preferably, the administration is by substantially selectively delivering the active compound, for example, so that at least 70% by weight of the active compound by-passes the oral cavity and is delivered onto the pharyngeal surfaces of the human.

Preferably, the administration is by use of a metered-dose dispenser with an adapter to substantially selectively deliver the active compound onto pharyngeal surfaces of the human, for example, so that at least 70% by weight of the active compound by-passes the oral cavity and is delivered onto the pharyngeal surfaces of the human.

Preferably, the adaptor is a mouthpiece-spacer attachment.

Preferably, the adaptor is a mouthpiece-spacer attachment having a length from 0.5 inch (1.27 cm) to 4.0 inches (10.2 mm).

Preferably, the active compound is delivered as a component of an aerosol. Preferably, the active compound is delivered in a unit dose.

Preferably, the unit dose is 2 to 10 mg of the active compound. Preferably, the unit dose is derived from 0.05 to 0.2 mL of a liquid formulation of the active compound.

### Medical Use

Another aspect of the present invention relates to an active compound for use in a method of treatment, for example:
A method of treating sensory discomfort in the upper airways of a human.
A method of treating sensory discomfort in the oropharynx of a human.
A method of alleviating pain from pharyngitis in a human.
A method of alleviating cough in a human.
A method of ameliorating the symptoms and signs of asthma, dyspnea, sleep apnea, snoring, or chronic obstructive pulmonary disease in a human.

Preferably, the treatment is by delivery of the active compound, for example:
to the oropharynx of the human.
more preferably, onto the oropharyngeal surfaces of the human.
still more preferably, to the lower retropalatal oropharynx (LRO) of the human.

Preferably, the treatment is by substantially selective delivery of the active compound, for example, so that at least 70% by weight of the active compound by-passes the oral cavity and is delivered onto the pharyngeal surfaces of the human.

Preferably, the treatment employs a metered-dose dispenser with an adapter to substantially selectively deliver the active compound onto pharyngeal surfaces of the human, for example, so that at least 70% by weight of the active compound by-passes the oral cavity and is delivered onto the pharyngeal surfaces of the human.

Preferably, the adaptor is a mouthpiece-spacer attachment.

Preferably, the adaptor is a mouthpiece-spacer attachment having a length from 0.5 inch (1.27 cm) to 4.0 inches (10.2 mm).

Preferably, the treatment is by delivery of the active compound as a component of an aerosol.

Preferably, the treatment is by delivery of the active compound in a unit dose.

Preferably, the unit dose is 2 to 10 mg of the active compound.

Preferably, the unit dose is derived from 0.05 to 0.2 mL of a liquid formulation of the active compound.

### Devices and Dispensers

Another aspect of the present invention relates to devices and dispensers charged with an active compound, and suitable for delivery of the active compound, for example:
to the oropharynx of a human.
more preferably, onto the oropharyngeal surfaces of a human.
still more preferably, to the lower retropalatal oropharynx (LRO) of a human.

Preferably, the device or dispenser is suitable to substantially selectively deliver the active compound, for example, so that at least 70% by weight of the active compound by-passes the oral cavity and is delivered onto the pharyngeal surfaces of the human.

Preferably, the device or dispenser is a metered-dose dispenser with an adapter to substantially selectively deliver the active compound onto pharyngeal surfaces of the human, for example, so that at least 70% by weight of the active compound by-passes the oral cavity and is delivered onto the pharyngeal surfaces of the human.

Preferably, the adaptor is a mouthpiece-spacer attachment.

Preferably, the adaptor is a mouthpiece-spacer attachment having a length from 0.5 inch (1.27 cm) to 4.0 inches (10.2 mm).

Preferably, the device or dispenser is adapted to deliver the active compound as a component of an aerosol.

Preferably, the device or dispenser is adapted to deliver the active compound in a unit dose.

Preferably, the unit dose is 2 to 10 mg of the active compound.

Preferably, the unit dose is derived from 0.05 to 0.2 mL of a liquid formulation of the active compound.

Optionally, the device or dispenser is accompanied by instructions (e.g., written instructions) regarding its use.

### Filling and Charging Devices and Dispensers

Another aspect of the present invention relates to a methods of preparing devices and dispensers charged with an active compound, comprising filling or charging the device or dispenser with a formulation comprising the active compound.

Preferably, the device or dispenser is suitable for delivery of the active compound, for example:
to the oropharynx of a human.
more preferably, onto the oropharyngeal surfaces of a human.
still more preferably, to the lower retropalatal oropharynx (LRO) of a human.

Preferably, the device or dispenser is suitable for substantially selectively delivering the active compound, for example, so that at least 70% by weight of the active compound by-passes the oral cavity and is delivered onto the pharyngeal surfaces of the human.

Preferably, the device or dispenser is a metered-dose dispenser with an adapter to substantially selectively deliver the active compound onto pharyngeal surfaces of the human, for example, so that at least 70% by weight of the active compound by-passes the oral cavity and is delivered onto the pharyngeal surfaces of the human.

Preferably, the adaptor is a mouthpiece-spacer attachment.

Preferably, the adaptor is a mouthpiece-spacer attachment having a length from 0.5 inch (1.27 cm) to 4.0 inches (10.2 mm).

Preferably, the formulation is suitable for generating an aerosol comprising the active compound as a component.

Preferably, the device or dispenser is adapted to deliver the active compound as a component of an aerosol.

Preferably, the device or dispenser is adapted to deliver the active compound in a unit dose.

Preferably, the unit dose is 2 to 10 mg of the active compound.

Preferably, the unit dose is derived from 0.05 to 0.2 mL of a liquid formulation of the active compound.

### Formulations

Another aspect of the present invention relates to a formulation comprising the active compound, and suitable for generating an aerosol comprising the active compound as a component.

Another aspect of the present invention relates to methods of preparing formulations which are suitable for generating an aerosol comprising the active compound as a component, comprising the step of admixing the active compound with one more suitable vehicles.

Examples of preferred vehicles include solvents, co-solvents, propellants, dispersing agents, carriers, and excipients. Examples of preferred solvents include purified water. Examples of preferred propellants including hydrofluorocarbons.

### The Active Compound

The active compound is a compound of Formula 1:

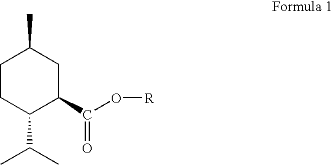

In one embodiment, —R is C₂ to C₄ hydroxyalkyl or polyhydroxyalkyl.

In one embodiment, —R is C₂ to C₄ hydroxyalkyl.

In one embodiment, —R is —R^{L1}—OH, wherein —R^{L1}-is saturated aliphatic C₂₋₄alkylene.

In one embodiment, —R is —CH₂CH₂OH, —CH₂CH₂CH₂OH, or —CH₂CH₂CH₂CH₂OH.

In one embodiment, —R is —CH₂CH₂OH.

In one embodiment, —R is C₂ to C₄ polyhydroxyalkyl.

In one embodiment, —R is —R^{L2}(OH)₂, wherein —R^{L2}< is saturated aliphatic C₂₋₄alk-tri-yl.

In one embodiment, —R is —CH₂CH(OH)CH₂OH.

### DETAILED DESCRIPTION

The inventor has found that topical and focused delivery of the active ingredient onto a discrete section of the oropharynx is sufficient to control cough. The total surface area of this cylindrical funnel is small, about the size of a US 25-cent to 50-cent piece, depending upon the age of the subject. The neurophysiological mechanism of drug action is indirect. The applied drug agent generates a signal from the lining of the throat that enters the brainstem and suppresses other irritative signals that enter the brain to initiate cough. This mechanism is called “gating” the signals that cause cough.

Because of the small target site, the therapeutically effective dose of the active ingredient is ideally delivered in a liquid volume of 0.05 to 0.2 mL, focused on the lower retropharyngeal surface using, for example, a disposable unit dispenser, a metered-dose inhaler device, a nebulizer with a mouthpiece attachment, etc. The mouthpiece attachment reduces unwanted delivery of the active ingredient to the tongue and buccal surfaces.

By this localized delivery method, the active ingredient acts on target receptors of the oropharynx, and the desired result of prolonged and effective cough or pain suppression is achieved. At single doses of 2 to 10 mg of the active compound, at a volume of 0.05 to 0.2 mL (e.g., 0.1 mL) per delivery unit, the delivered dose has a rapid onset of relieving sensory discomfort in less than 1 minute. The active compound acts indirectly to gate nociception, to soothe the throat, and to have potent anti-tussive action exceeding several hours.

### Cold and Coolness Receptors

Approximately 200 million years ago, certain living organisms acquired the ability to control metabolic heat production (endothermy) and to maintain a constant internal body temperature (homeothermy). This evolutionary transition, from a “cold-blooded” to a “warm-blooded” physiology, enabled such species to better adapt and survive in a variable environment. Associated with this change was the development of sensory systems for monitoring temperature, especially in the upper respiratory tract and the skin, in order to control body temperature. Coolness, the first signal to warn about the need for heat conservation, is a pervasive neuronal signal from the organism's surfaces such as the eyes, face, nose, ears, and neck. For example, from the skin of the face of humans, about 92% of the thermoceptive input is from cold neurons that are tonically active at about 18° C.

The ability of coolness/cold to reduce sensory discomfort from the body's surfaces (irritation, itch, and pain) is a common experience. Thus, air-conditioning, cold water, and ice, can be used to relieve sensory discomfort and the heat withdrawal necessary for coolness/cold can be achieved with gas, liquid, or solid materials. Surprisingly, it has been scarcely recognized that activation of the coolness/cold neuronal pathways with specific chemical agents has the potential for remarkable therapeutic benefit. Several new insights help bring this idea forward.

Coolness/cold produced by chemical agents reduces sensory discomfort by centrally “gating” the flow of nociceptive information. The mode of action is indirect: that is, there is no interference with the generation, transmission, or flow of the input of nociceptive information into the central nervous system. This is by contrast to the actions of heat-withdrawing methods such as ice which may, in part, reduce the pain of injured tissues by local decrease of tissue metabolism or by inhibiting blood flow. The precise neurotransmitter circuitry which underlies the gating process of chemical agents is not known, but the sheer dominant volume of coolness/cold signals may be sufficient to over-ride nociceptive signals.

In the skin, the area innervated by a single spinal nerve is a called a dermatome. Sensations from immediately adjacent dermatomes can influence each other because there is overlap in the somatotopic organization of the sensory projections. A good example of such phenomenon is scratching and itch. Mechano-receptors can be activated by scratching to reduce itch, but it is not necessary to precisely scratch the point source of the itch. Adjacent sites will also suffice. Similarly, the sensory information from afferents in the upper airways and viscera also converges in the brainstem.

In the present invention, the focus is on the sensory input from two cranial nerves, the glossopharyngeal (9^{th} nerve) and the vagus (10^{th}). The glossopharyngeal nerve carries sensory information from the oropharynx into the brainstem. The vagus carries information from the larynx and the upper esophagus into the brainstem. The afferent signals from the two cranial nerves converge in the spinotrigeminal tract of the brainstem.

Nociceptive signals in the larynx gives rise to cough and signals from the esophagus may contribute to non-cardiac pain. These signals come principally from the vagus.

The present invention relates to the application of a chemical cooling agent onto the oropharyngeal surface, which sends, via the glossopharyngeal nerve, a signal into the brainstem that will indirectly “gate” the nociceptive signals from the vagus and thus achieve the therapeutic goals of reducing cough and pain.

### The Active Compounds

The inventor has found that certain p-menthane-carboxylic acid hydroxy (or polyhydroxy) esters of Formula 1 (as described herein) have the desirable quality of cooling and soothing actions on the throat when applied onto the lower retropalatal surface of the pharynx.

Additionally, these compounds are odorless liquids and are not irritating to the tissues lining the mouth or throat. These compounds, when applied at a volume of 0.05 to 0.2 mL to deliver a dose of 2 to 8 mg of the active ingredient, suppress cough and pain.

Surprisingly, in therapeutic situations, after the initial sensations of coolness and refreshment from these compounds have dissipated, there is prolonged relief. For example, in a subject with non-productive smoker's cough, a single application of an active compound suppressed coughing for 3 to 5 hours. After several repeated applications, the subject no longer complained of or manifested coughing.

Examples of p-menthane-carboxylic acid hydroxy (or polyhydroxy) esters of Formula 1 (as described herein) were first described by Watson et al., 1977, U.S. Pat. No. 4,033,994.

Compounds CPS-004 and CPS-030 both have good cooling qualities.

### Targeted Drug Delivery

The pharynx is divided into three regions: naso, oro and laryngo. The nasopharynx, also called the rhinopharynx, lies behind the nose and above the level of the soft palate. The oropharynx reaches from the soft palate to the level of the hyoid bone. The laryngopharynx reaches from the hyoid bone to the lower border of the cricoid cartilage, where it is continuous with the esophagus. The oropharynx may be further divided into an upper and lower region, the mid-point being what is called the lower retropalatal oropharynx (LRO). See, for example, the magnetic resonance imaging studies shown in Daniel et al. (“Pharyngeal dimensions in men and women.” Clinics vol. 62, pg. 5-10, 2007). The pharynx is a funnel shaped tube and the LRO is the narrowest point with the smallest measured diameter (typical dimensions: lateral-latero, 1.8 cm and anterior-posterior, 0.58 cm). In the present invention, this area of about 3 to 5 cm² is the preferred target for drug delivery.

One method to reach this target area is to administer the active ingredient in a rapidly-disintegrating tablet. See, for example, Wei, 2006, WO 2006/103401 A2.

A preferred method is to use a metered-dose dispenser with a mouthpiece attachment to by-pass the oral cavity absorption sites, and to deliver the active ingredient onto the LRO. The metered-dose dispenser is a device familiar to those skilled in the art and consists of a formulation, valve and container, and actuator.

Two types of dispensers are in wide use for the treatment of asthma and chronic obstructive pulmonary disease (COPD). The metered-dose inhaler (MDI) has an active ingredient formulation in a haloalkane vehicle, usually a fluorinated alkane, stored in a metal container under pressure, and the actuator releases the contents as an aerosol into a short (0.75 to 1.5 inch) mouthpiece. The nebulizer type of dispenser passes compressed air through a reservoir of liquid drug formulation usually stored in a transparent glass bottle. The aerosol generated by mixing the air and liquid is then passed through an aperture of precise design into a mouthpiece and attachment that is usually several inches long. These dispensers differ from the throat spray type of delivery system where there are no mouthpieces or attachments.

The design of metered-dose dispensers can accurately control volume of delivery and the particle size distribution of the emitted aerosol. These dispensers are designed primarily to deliver drugs, e.g., β-adrenoceptor agonists (e.g., albuterol), muscarinic receptor antagonists (e.g., ipratropium), and glucocorticosteroids (e.g., fluticasone proprionate), onto the surfaces of the bronchi and bronchioles for the treatment of asthma and COPD. Thus, formulations are chosen to avoid pharyngeal deposition and to favor deposition on the surfaces of the bronchi and bronchiole. This can be accomplished by adding dispersing agents to the formulation which reduces particle agglomeration or coalescence or by decreasing the hypophilic properties of the excipients: the net effect being a decrease in the aerosol particle size and the ability of the particle to remain in suspension.

For the treatment of asthma and COPD, the preferred ratio of respirable particle fraction/throat deposition fraction is 70%/30%. By contrast, the design of the dispenser for the practice of the present invention is the reverse, with the goal of ≧70% throat deposition, to maximize oropharynx deposition, yet by-passing deposition on the tongue and buccal surfaces. Thus, particle size distributions of ≧5 μm mass median diameter are ideal because the dose will be deposited on the oropharynx surface and not into the lower respiratory tract.

This use of metered-dose dispensers for deliberate pharyngeal delivery of an active ingredient has not been previously contemplated.

Current methods for delivery of anti-cough drugs do not recognize the specific importance of focused delivery of the active ingredient to the LRO. See, e.g., Sixsmith, 1994, U.S. Pat. No. 5,846,557; and Eisenstadt et al., 1998, U.S. Pat. No. 5,846,557.

Lozenges and chewing gum when placed in the oral cavity release active ingredients which dissolve in the mouth and are diluted by saliva. This mixture is swallowed so the overall contact time with the LRO is limited. These delivery methods result in drug effects on and absorption of drug from the buccal lining, both events which may not be desirable. The same limitations apply to a throat spray which is an inefficient delivery method. A cooling agent delivered by a throat spray into the mouth will mainly be deposited on the tongue and the lining of the mouth and activate sensory pathways in the trigeminal (5^{th}) nerve. The central convergence of the trigeminal nerve is somatotopically more distant from the vagal inputs. Thus, these drug delivery methods have limited efficacy in the treatment of cough.

Ideally, delivery of a bolus of drug focused on the LRO is the best method of practice. To achieve this objective, drug delivery to the LRO is accomplished with dispensers with attachments designed for such purpose. A mouthpiece-spacer attachment differs from a spray device in that the lips are closed around the outlet of the dispenser whereas with a spray the mouth is held open and the spray is focused on the surface of the tongue. Images and examples of such dispensers with mouthpiece and mouthpiece attachments are, for example, found in the web pages of Pari Respiratory Co. (www.pari.com), a major manufacturer of respiratory equipment. As can be seen in the images, the mouthpieces of standard metered-dose inhalers (MDI) are usually 0.5 to 1.5 inches (1.25 to 3.8 cm) long. For nebulizers (delivery systems with a compressed air pump and a bottled reservoir of drugs), the mouthpiece may be 2 to 5 inches (5.1 to 12.7 cm) long. The term “spacer” is used to describe attachments to the mouthpiece of MDIs that facilitate mixing of the emitted aerosol and inhalation of the aerosol.

In the present invention, the outlet from the reservoir container should be at 0.5 inches (1.27 cm) long and not more than 4.0 inches (10.2 mm). This length would be sufficient for individual taking the medication to enclose their lips around the outlet. This delivery mechanism allows the active ingredient to by-pass the oral cavity and to be focally delivered onto the pharyngeal surfaces. The degree of emitted pressure, mixing of the active ingredients with air, formulation and excipients, valve dimensions, and use of mouthpiece and attachments are then designed to favor the preferred throat deposition fraction of ≧70%. The phrase “substantially selective delivery of an aerosol by the dispenser” in intended to reflect this preference.

Examples of suitable delivery devices include, e.g., Atrovent® HFA Inhalation Aerosol from Boehringer Ingelheim; Ventrolin® Evohaler from Glaxo SmithKline; and Vortex® from PARI Respiratory. An example of a suitable commercially available spacer is Aerochamber®.

It should be noted, however, in some clinical conditions, that a smaller particle size range, with the goal of delivery of compounds of Formula 1 into the bronchi and bronchioles is also contemplated. These conditions are those in which there is substantial irritation of the surfaces of the bronchi and bronchioles, such as occurs in asthma and chronic obstructive lung diseases (COPD). A cooling agent does have effect on the sensory elements in the airways, as witnessed by the use of mentholated cigarettes. The presence of menthol in cigarettes reduces the irritation produced by the smoke and thus allows deeper inhalation and uptake of nicotine.

Mechanism of Action and Selection of Active Ingredient (−)-Menthol is used as a sensory agent in anti-cough lozenges. In the upper airways and oral cavity, (−)-menthol has multimodal action on sensations and can elicit somatosensation (cooling, irritation, tingling), olfaction (minty), and gustation (bitter). As a counter-irritant, (−)-menthol can reduce irritation of oral and pharyngeal membranes (e.g., strong mints or toothpastes) and have analgesic actions on muscle (e.g., BenGay® ointment). The multimodalities may further mix to give rise to complex perceptions of irritation (burning, prickling, stinging), especially around the eyes, of thermal effects (cooling, warming) and of tactile effects (buzzing, tingling, tickling, numbing). In the nose and oral cavity, the predominant mode of detecting (−)-menthol is olfactory (see, e.g., Nagata et al., 2005, J. Exptl. Psychol., Vol. 31, pp. 101-109).

The receptor for menthol and some cooling agents is thought to be a protein called TRP-M8, but menthol also acts on receptors called TRP-A1 and TRP-V3. The inventor has reported that a compound known as WS-12 is 2000 times more potent than menthol on TRP-M8. However, this compound does not produce very much cooling action on the skin. The inventor has reported that the potency of compounds that activate TRP-M8 is not correlated to cooling actions (see, e.g., Vogt-Eisele et al., 2005, “N-Alkylcarboxamide Cooling Agents: Activities on Skin and Cells with TRPM8 and TRPA1 Receptors”, 3rd Annual Workshop on the Study of Itch, Sep. 25 to 27, 2005 in Heidelberg, Germany, Acta Dermato-Venereological, Vol. 85, p. 468). Furthermore, TRP-M8 is activated by mustard oil, an agent that produces the pungent sensations of wasabi.

Thus, compounds described in this invention cannot be viewed solely as menthol-like or acting via TRP-M8 receptors.

The compounds of Formula 1 (described herein) clearly differ from (−)-menthol because they are non-volatile, lack odor, lack irritancy, and are liquids (at room temperature) that can be miscible in a solvent to be delivered to the LRO. The compounds are active at single doses of less than 10 mg, and in most cases, 2 to 5 mg per dose is sufficient for anti-tussive activity. Additionally, the compounds are characterized by rapid onset of action, on the order of 0.5 to 2 minutes. As such, they represent a novel and unexpected class of compounds with cough suppressant and anti-nociceptive effects on the airways.

Two preferred compounds of Formula 1 are shown below:

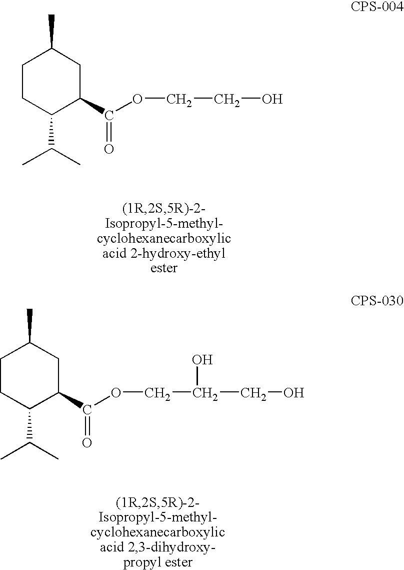

These compounds are liquids at room temperature and therefore suitable for incorporation into solvents used for metered-dose dispensers.

Activation of sensory afferents in the oropharynx by compounds of Formula 1 (as described herein) are believed to initiate sensory signals that over-ride of “gate” the signals for the cough reflex. A gating mechanism has been suggested by Bromm et al. (see, e.g., Bromm et al., 1995, “Effects of menthol and cold on histamine-induced itch and skin reactions in man”, Neuroscience Letters, Vol. 187, pp. 157-160) for the effects of menthol on itch and by Proudfoot et al. (see, e.g., Proudfoot et al., 2006, “Analgesia mediated by TRP-M8 in chronic neuropathic pain”, Current Biology, Vol. 16, pp. 1591-1605) for the inhibitory effects of a compound called icilin on neuropathic pain, but the idea of “gating” of cough signals with a cooling agent has not been previously proposed.

The afferent signal from the oropharynx is via the 9^{th} cranial nerve (glossopharyngeal nerve) and the cough signals, thought to originate from the laryngopharynx, are carried by the 10^{th} cranial nerve (vagus nerve). For the compounds of Formula 1 (as described herein), it is believed that the mechanism of action is for the cooling signals from the nerve endings of the orpharynx to enter into the brainstem via the 9^{th} nerve and then “gate” the irritant cough signals coming into the brainstem via the 10^{th} nerve. This mode of action may be termed “indirect.”

The compounds of Formula 1 (as described herein) have one or more of all of the following desirable properties:
a therapeutically useful anti-tussive and anti-nociceptive effect that is readily obtained at a single dose of less than 10 mg when the compound is applied onto surfaces of the mouth and throat.
a duration of anti-tussive action that exceeds several hours when applied at a dose of 10 mg or less, and occurs even when other sensory effects, such as cooling, are no longer present. Furthermore, repeat applications do not result in de-sensitization to the anti-tussive effect.
a desirable sensory selectivity, that is, the sensations felt with these compounds in the throat were mainly coolness, soothing, and refreshing, and unlike menthol, without harsh taste, irritation, tingling, burn, or sting.
physicochemical properties of aqueous solubility, miscibility and solubility in fluoroalkane propellants, stability, and octanol/water partition coefficients that facilitate formulations for delivery.
an absence of odor or irritancy when the compounds contact the oral and airway surfaces.
a novel mechanism of drug action, not previously described for anti-tussive drug actions.
a good safety profile based on structural considerations of the compound.

The above properties of the active ingredient and the preferred method of delivery of compounds permit the construction of a patient-friendly medication. For example, individuals can be taught to use a hand-held metered-dose dispenser or a nebulizer with a neck (e.g., a 2.5 inch (6.3 cm) neck), to deliver a fixed amount (e.g., 0.1 mL) to the back of the mouth. Currently, there are no anti-tussive medications designed with such ease of use and fast-acting relief.

Formulation of Active Ingredient in the Reservoir

In formulating anti-tussive compositions for use according to the present invention, the active ingredient may be incorporated into a vehicle that by itself may be inert or may contain other active ingredients.

Suitable vehicles include the standard fluoroalkanes used in metered-dose inhalers and, for aqueous formulations, purified water. The hydrofluorocarbon propellant is preferably selected from the group of HFA 134a, HFA 227 and mixtures thereof. In aqueous hydrophilic formulations, the co-solvent is usually an alcohol, preferably ethanol or propylene glycol.

The low volatility component, when present, is selected from the group of glycols, particularly propylene glycol, polyethylene glycol and glycerol; alkanols such as decanol (decyl alcohol); sugar alcohols including sorbitol, mannitol, lactitol and maltitol, glycofural (tetrahydro-furfurylalcohol) and dipropylene glycol; alkanes, for example dodecane and octadecane; terpenes, for example menthol, eucalyptol, limonene; sugars, for example lactose, glucose, sucrose; polysaccharides, for example ethyl cellulose, dextran; antioxidants, for example butylated hydroxytoluene, butylated hydroxyanisole; polymeric materials, for example polyvinyl alcohol, polyvinyl acetate, polyvinyl pyrrolidone; amines, for example ethanolamine, diethanolamine, triethanolamine; steroids, for example cholesterol, cholesterol esters.

In addition, a dispersing agent may be required to disperse the particles and to maintain them in suspension. Commonly used dispersing agents include oleic acid, sorbitan oleate, sorbiton trioleate, sorbiton sesquioleate and lethicin. By maintaining the particles in suspension, dispersing agents help to ensure that a uniform dose is dispensed each time the metered valve is depressed. The aerosol compositions to be delivered with the pressurised MDIs may contain from 0.2 to 2% by weight of said low volatility component.

A typical diluent, carrier, or vehicle in an aqueous formulation may be a “polyhydric alcohol”, for example, xylitol, mannitol, sorbitol, maltitol, isomaltitol, maltotriitol, lactitol, or β-linked-glucopyranasido-sorbitol. These liquids, after sterilization by filtration, may be combined with preservatives, flavoring agents, solvents, and then dispensed from a reservoir type of storage container or from unit dose containers such as are readily available commercially. Flavoring agents such as the sweeteners, chocolate, aspartame, sucralose, or saccharin, may be added to mask any undesirable tastes. These common additives are familiar to those skilled in the art.

A 20 to 50 mg/mL dose of a compound of Formula 1 (as described herein) in formulations according to the present invention is a particularly preferred concentration for delivery of a unit dose of about 0.1 mL.

### Safety Profile of Active Ingredients

The compounds of Formula 1 (as described herein) may conveniently be considered to be comprised of two moieties: a (1R,2S,5R)-2-isopropyl-5-methylcyclohexane carboxylic acid conjugated to a diol, e.g., 1,2-ethanediol, or a polyol, e.g., glycerol.

The first component, known as compound WS-1, is encountered in the context of a compound known as WS-3 (the ethylamide derivative of WS-1), which is a cooling agent permitted for use in cosmetics, toiletries and confectionery. The pathways for the metabolism of diols and polyols are known, and, at topical doses of less than 10 mg, is unlikely to be of toxicological significance. Thus, the compounds of Formula 1 are believed to have a good safety profile.

### EXAMPLES

### Example 1

### Synthesis of P-Menthoyl Carboxylic Acid Hydroxy-Esters

### 1R,2S,5R)-2-Isopropyl-5-methyl-cyclohexanecarboxylic acid 2-hydroxy-ethyl ester CPS-004

### 1R,2S,5R)-2-Isopropyl-5-methyl-cyclohexanecarboxylic acid 2,3-dihydroxy-propyl ester CPS-030

The synthesis of compounds of Formula 1 is familiar to those of skill in the art of chemistry and commences with the reaction of an appropriate alkyl-substituted-cycloalkyl-carboxylic acid with thionyl chloride to form the carbonyl chloride. This latter is then reacted with an excess of the appropriate alcohol derivative, optionally in the presence of a suitable hydrogen chloride acceptor, or base. Many suitable alcohols may be obtained from commercial sources such Sigma-Aldrich Co., St. Louis, Mo. Suitable procedures are described in Watson et al., 1977, U.S. Pat. No. 4,033,994. For example, to synthesize CPS-004, 5-methyl-2-isopropylcyclohexane carboxylic acid is prepared by reaction of the corresponding alcohol, (−)-menthol, with zinc chloride in hydrochloric acid to form 5-methyl-2-isopropylcyclohexyl chloride. From this, the Grignard reagent is generated in dry tetrahydrofuran (THF), followed by carbonation with gaseous carbon dioxide to form the carboxylic acid. The carboxylic acid is then converted into its acid chloride, for example, by reaction with thionyl chloride. The acid chloride is then reacted with the appropriate alcohol, for example, 1,2-ethanediol or glycerol, to form the corresponding ester as a colorless liquid.

### Example 2

### Octanol/Water Partition Coefficients

The log P (octanol/water partition coefficient) of the compounds of Formula 1 were calculated and are shown in the Table. It can be seen that these compounds are compatible with incorporation into a vehicle and stored in a reservoir for metered-dose delivery with an aerosol dispenser.

The log P values also give parameters for selection of the principal vehicle and co-solvents. For example, CPS-160 has exceptional aqueous solubility with a low log P value and should be compatible with an aqueous solvent containing 1 to 5% of ethanol or propylene glycol. On the other hand, for longer term reduction of sensory irritation in the airways, such as use in COPD, compound CPS-003 may be a better choice.

TABLE 1
Partition Coefficient Data

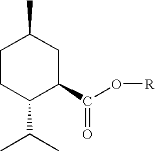

| | | | | | | | | Calculated |
|---|---|---|---|---|---|---|---|---|
| | | | | | | | | Water |
| | | | | | | | | Solubility |
| | —R | LogP | S.D. | LogP | S.D. | LogP | S.D. | (mmol/L) |
| 1* | —CH₂CH₂OH | 2.82 | 0.47 | 2.68 | 0.49 | 2.27 | 0.47 | 5.02 |
| 2 | —CH₂CH₂CH₂OH | 2.93 | 0.47 | 2.74 | 0.49 | 2.73 | 0.48 | 0.46 |
| 3 | —CH₂CH₂CH₂CH₂OH | 3.38 | 0.47 | 3.19 | 0.49 | 3.18 | 0.49 | 0.14 |
| 4 | —CH₂CH₂OCH₂CH₂OH | 2.66 | 0.47 | 2.52 | 0.49 | 2.19 | 0.58 | 4.22 |
| 5** | —CH₂CH(OH)CH₂OH | 2.28 | 0.47 | 2.31 | 0.49 | 1.36 | 0.63 | 101 |
| 6 | —CH(CH₃)CH₂OH | 3.14 | 0.47 | 3.10 | 0.49 | 2.86 | 0.63 | 3.15 |
| 7 | —CH₂CH(OH)CH₃ | 3.14 | 0.47 | 3.10 | 0.49 | 2.53 | 0.57 | 3.15 |

*CPS-004,
**CPS-030

### Example 3

### Bioassay

CPS-004 and CPS-030 are liquids at room temperatures. These compounds were tested by placing 0, 2, 5 and 8 mg on a 6-inch glass rod and applying these compounds onto the posterior third of the dorsal surface of the tongue. After depositing the test substance, the subject was instructed to keep the mouth closed and allow the test substance to distribute to the back of the mouth (orpharynx). The presence and duration of sensations from the oral cavity was then noted.

Substances at 2 mg were noted as having a cooling effect on the back of the mouth. CPS-004 and CPS-030 had similar potency. CPS-030 had an acrid taste which was objectionable. The duration of cooling lasted for 10 to 15 minutes, with the higher doses producing a cooling sensation that could still be detected after 30 minutes.

In the next set of experiments, CPS-004 or CPS-030 was mixed 50 mg/mL in a solution of 5% ethanol-95% physiological saline. 10 mL each of solution was then separately placed in a reservoir which was part of a hand-activated pressurized metered-dose dispenser, with and without attachment to a 1.5 inch (3.8 cm) PE50 plastic tubing attached to the outlet of the dispenser. Each compression delivered 0.1 mL of the stored solution.

Five subjects then tested the emitted solution. Without the attachment, the output was deposited mainly on the surface of the anterior tongue. Cooling sensations were felt primarily on the tongue surface. With the attachment, cooling sensations were localized to the back of mouth in the lower retropalatal oropharynx (LRO). Here, the sensations were much stronger and focused. The duration of cooling ranged from 10 to 25 minutes.

### Example 4

Two subjects with cough volunteered to test the CPS-030 solution. One of the subjects had a persistent cough from chronic asthma and one subject had a chronic cough from seasonal allergy to grass pollen. Application of the CPS-030 solution with the attachment was effective in stopping the cough for 3 to 4 hours, but no beneficial effects were obtained without the attachment. Subjects could titrate their sense of throat irritation to control cough and repeat doses did not result in any de-sensitization to the anti-cough effect. The subjects remarked on the significant relief that was obtained and the ability to go to sleep without being disturbed by coughing.

Two other individuals with pain and hoarseness from pharyngitis (one due to her job as a tour guide) also tested the CPS-030 solution. Both reported relief from throat discomfort within a few minutes after applying 0.1 mL of the solution with an attachment. One subject, who previously could not speak comfortably, now managed to conduct a conversation for one hour afterwards.

In summary, it was revealed here that several cooling agents that are liquids at room temperatures and originally synthesized in 1977 for possible use in toiletries have therapeutic benefit in the treatment of an irritated pharyngeal lining if delivery of the agent can be topically localized to a specific area of the pharynx using, for example, a metered dose device adapted for such a purpose. As current medications for treatment of an irritated pharynx (in conditions such as cough) have limited effectiveness, the method described here represents innovative technology.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
Bromm et al., 1995, “Effects of menthol and cold on histamine-induced itch and skin reactions in man”, Neuroscience Letters, Vol. 187, pp. 157-160.
Cherukuri et al., 2000, “Soft and Chewy Cough and Cold Relief Composition”, international patent publication number WO 00/37044 A1 published 29 Jun. 2000.
Daniel et al., 2007, “Pharyngeal dimensions in men and women”, Clinics, Vol. 62, pp. 5-10.
Eisenstadt et al., 1998, “Chewing gum containing cough suppressing agent”, U.S. Pat. No. 5,846,557 granted 8 Dec. 1998.
Nagata et al., 2005, “qqq title”, J. Exptl. Psychol., Vol. 31, pp. 101-109.
Pan et al., 1999, “Delivery system for the localized administration of medicaments to the upper respiratory tract and methods for preparing and using same”, U.S. Pat. No. 5,912,007, granted 15 Jun. 1999.
Proudfoot et al., 2006, “Analgesia mediated by TRP-M8 in chronic neuropathic pain”, Current Biology, Vol. 16, pp. 1591-1605)
Sixsmith, 1994, “Pharmaceutical lozenges”, U.S. Pat. No. 5,322,694 granted 21 Jun. 1994.
Vogt-Eisele et al., 2005, “N-Alkylcarboxamide Cooling Agents: Activities on Skin and Cells with TRPM8 and TRPA1 Receptors”, 3rd Annual Workshop on the Study of Itch, Sep. 25 to 27, 2005 in Heidelberg, Germany, Ada Dermato-Venereological, Vol. 85, p. 468.
Watson et al., 1977, “Substituted p-menthanes”, U.S. Pat. No. 4,033,994, granted 5 Jul. 1977.
Wei, 2006, “N-Alkylcarbonyl-Amino Acid Ester and N-Alkylcarbonyl-Amino Lactone Compounds and Their Use”, international patent publication number WO 2006/103401 A2.

1-6. (canceled)

70. A method for treatment of sensory discomfort in the upper airways or oropharynx, alleviating pain from pharyngitis, alleviating cough, or ameliorating the symptoms and signs of asthma, dyspnea, sleep apnea, snoring, or chronic obstructive pulmonary disease in a human, comprising administering to said human a therapeutically-effective amount of a compound of formula 1, 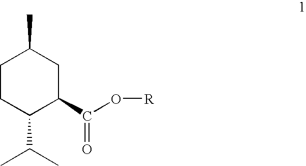
wherein R is C₂ to C₄ hydroxyalkyl or polyhydroxyalkyl, and
wherein the compound is administered by delivery to the oropharynx of the human.

71. The method according to claim 70, wherein the compound is delivered onto the oropharyngeal surfaces of the human.

72. The method according to claim 70, wherein the compound is delivered to the lower retropalatal oropharynx of the human.

73. The method according to claim 70, wherein the treatment is by substantially selective delivery of the compound.

74. The method according to claim 70, wherein the treatment is by substantially selective delivery of the compound so that at least 70% by weight of the compound by-passes the oral cavity and is delivered onto the pharyngeal surfaces of the human.

75. The method according to claim 70, wherein the treatment employs a metered-dose dispenser with an adapter to substantially selectively deliver the compound onto pharyngeal surfaces of the human.

76. The method according to claim 70, wherein the treatment employs a metered-dose dispenser with an adapter to substantially selectively deliver the compound onto pharyngeal surfaces of the human so that at least 70% by weight of the compound by-passes the oral cavity and is delivered onto the pharyngeal surfaces of the human.

77. The method according to claim 70, wherein the treatment is by delivery of the compound as a component of an aerosol.

78. The method according to claim 70, wherein the treatment is by delivery of the compound in a unit dose.

79. The method according to claim 78, wherein the unit dose is 2 to 10 mg of the compound.

80. The method according to claim 79, wherein the unit dose is derived from 0.05 to 0.2 mL of a liquid formulation of the compound.

81. The method according to claim 70, wherein the compound is selected from (1R,2S,5R)-2-isopropyl-5-methyl-cyclohexanecarboxylic acid 2-hydroxy-ethyl ester and (1R,2S,5R)-2-isopropyl-5-methyl-cyclohexanecarboxylic acid 2,3-dihydroxy-propyl ester.

82. A device or dispenser charged with a compound of formula 1 and suitable for delivery of the compound to the oropharynx of a human: 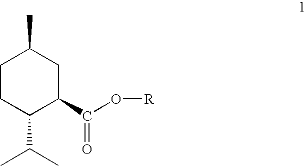
wherein R is C₂ to C₄ hydroxyalkyl or polyhydroxyalkyl.

83. A formulation comprising a compound of formula 1, wherein the formulation is suitable for generating an aerosol comprising the compound as a component: 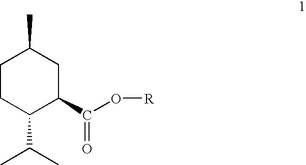
wherein R is C₂ to C₄ hydroxyalkyl or polyhydroxyalkyl.

84. A metered dose dispenser adapted for pharyngeal delivery of an active ingredient.

85. The metered dose dispenser according to claim 84 comprising an aerosol formulation adapted for delivery of said active ingredient to the oropharynx surface.
